(19) United States
(12) Patent Application Publication
(10) Pub. No.: US 2026/0061206 A1
(43) Pub. Date: Mar. 5, 2026

(54) INTELLIGENT CPR DEFIBRILLATOR, AND SYSTEM AND METHOD FOR AUTOMATICALLY SENDING TIME NODES THEREOF

(71) Applicant: SIR RUN RUN SHAW HOSPITAL ZHEJIANG UNIVERSITY SCHOOL OF MEDICINE, Hangzhou (CN)

(72) Inventor: Qin JIANG, Hangzhou (CN)

(21) Appl. No.: 18/901,268

(22) Filed: Sep. 30, 2024

(30) Foreign Application Priority Data
Aug. 28, 2024 (CN) 202411187938.9

(51) Int. Cl. A61N 1/39

(52) U.S. Cl. CPC A61N 1/39044; A61N 1/3925

(57) ABSTRACT

An intelligent cardiopulmonary resuscitation (CPR) defibrillator, and a system and a method for automatically sending time nodes thereof, related to a field of defibrillator devices are provided. The intelligent CPR defibrillator includes a constructing component. The constructing component includes a constructing part, an unfolding part provided at a first side of the constructing part, and at least one clamping part provided at a second side of the constructing part. The constructing part includes a storage box and a defibrillator body provided in the storage box. The system includes a sensor module, a processor module, a mechanical arm module, a defibrillator module, and a communication module.

### TECHNICAL FIELD

The present disclosure relates to a field of defibrillator devices, and in particular to an intelligent cardiopulmonary resuscitation (CPR) defibrillator, and a system and a method for automatically sending time nodes thereof.

### BACKGROUND

Sudden cardiac arrest is an emergency medical condition requiring immediate cardiopulmonary resuscitation (CPR) to improve a survival rate of patients. Conventional CPR relies on manual operation, which not only requires operators to have professional medical skills, but also demands high accuracy and better continuity of operation. However, in an emergency, timely finding qualified operators is not easy, and skill levels of different operators may significantly affect quality and effect of artificial CPR.

Currently, some automatic CPR devices are capable of simulating a process of the artificial CPR to a certain extent. However, such automatic CPR devices are hard to automatically judge and adjust force and frequency of the CPR according to specific conditions of the patients due to a lack of intelligent judgement mechanisms.

Electrocardiogram (ECG) signals are important indicators for assessing heart conditions. Conventional electrocardiogram signal analysis relies on subjective judgment of doctors and may not be handles in time during emergencies. Although automatic ECG signal analysis technologies are capable of providing rapid analysis of heart rates and heart rhythms, the automatic ECG signal analysis technologies still lacks comprehensive evaluation of irregularity of the heart rhythms, so that there are limitations on applications of the automatic ECG signal analysis technologies in automatic CPR decision-making.

Therefore, those who skilled in the art put forward an intelligent CPR defibrillator, and a system and a method for automatically sending time nodes thereof to address the problems raised herein.

### SUMMARY

In order to solve above technical problems, the present disclosure provides an intelligent cardiopulmonary resuscitation (CPR) defibrillator, and a system and a method for automatically sending time nodes thereof to solve the problems in the prior art.

The present disclosure provides the intelligent CPR defibrillator, including a constructing component. The constructing component includes a constructing part, an unfolding part provided at a first side of the constructing part, and at least one clamping part provided at a second side of the constructing part. The constructing part includes a storage box and a defibrillator body provided in the storage box. The at least one clamping part includes a sliding rod slidably provided at a first side of the storage box, and a clamping plate provided at one end of the sliding rod.

Furthermore, the unfolding part includes a driving gear rotatably provided at a second side of the storage box, a driven gear rotatably provided at a first side of the driving gear, and a rotating rod provided at a second side of the driving gear, the driving gear is meshed with the driven gear. The unfolding part further includes first connecting rods respectively hinged at a third side of the driving gear and a first side of the driven gear, at least one first placing box provided at a third side of the storage box, a second placing box provided at one side of the at least one first placing box, and second connecting rods provided at the second side of the storage box, the second connecting rods are respectively connected with a side wall of the storage box, a side wall of the at least one first placing box, and a side wall of the second placing box.

Furthermore, the at least one clamping part further includes a protective pad provided at one end of the clamping plate, a fixing plate provided at one end of the sliding rod, and a return spring sleeved on an outer side of the sliding rod. the at least one clamping part further includes a toggle rod provided at the first side of the storage box.

Compared with the prior art, the present disclosure has following beneficial effects.

The present disclosure provides the constructing component including the constructing part, the unfolding part provided at the first side of the constructing part, and the at least one clamping part provided at the second side of the constructing part. The constructing component is put into the storage box for clamping and fixing when the intelligent CPR defibrillator is not in use, so as to improve protective effect of the storage box on the constructing component. When using the intelligent CPR defibrillator, the storage box is quickly opened, clamping on the constructing component is released during opening the storage box, and the constructing component is quickly got out of the storage box for use, thereby improving portability of the intelligent CPR defibrillator and reducing likelihood of collision, damage and fault resulted from environmental impact during using the intelligent CPR defibrillator outdoors.

In view of a problem that it is still difficult to automatically judge and adjust force and frequency of the CPR according to specific conditions of the patients in an actual use process, the present disclosure provides the system, suitable for automatically sending operation time nodes by the intelligent CPR defibrillator as foregoing and applied to the intelligent CPR defibrillator as foregoing, including a sensor module, a processor module, a mechanical arm module, a defibrillator module, and a communication module.

The sensor module is configured to detect electrocardiogram signals of a human body and includes at least one bioelectrical signal sensor, where the at least one bioelectrical signal sensor is configured to monitor a heart rate and a heart rhythm of human body in real time.

The processor module is configured to analyze the electrocardiogram signals and determine whether CPR is needed.

The mechanical arm module is configured to perform the CPR.

The defibrillator module is configured to perform automatic defibrillation.

The communication module is configured to record the operation time nodes and automatically send the operation time nodes to a remote monitoring system.

Furthermore, the processor module employs a following algorithm formula to analyze the electrocardiogram signals and determine whether the CPR is needed,

$$CPR\_Necessity = \{ \begin{matrix}True if Heart\_Rate < Threshold\_Low or Heart\_Rate > \\ Threshold\_High or Heart\_Rhythm \_Irregularity \\ > Threshold\_Irregularity \\ False otherwise\end{matrix} ,$$

the CPR_Necessity is a Boolean value (True or False) determining whether the CPR is needed according to a comparison result of irregularity of the heart rate with a threshold of the heart rate and a comparison result of irregularity of the heart rhythm with a threshold of the heart rhythm, the Threshold_LowThreshold_Low and the Threshold_HighThreshold_High are preset thresholds of the heart rate, the Heart_Rhythm_IrregularityHeart_Rhythm_Irregularity is a measure of the irregularity of the heart rhythm, and the Threshold_IrregularityThreshold_Irregularity is a threshold of the irregularity of the heart rhythm.

Furthermore, the mechanical arm module is configured to automatically adjust a position thereof and a force thereof according to an instruction from the processor module to implement effective CPR. The defibrillator module is configured to automatically adjust electric shock energy and perform electric shock when needing defibrillation.

Furthermore, the communication module is configured to send the operation time nodes to the remote monitoring system in real time in a format of

$$Time\_Stamp = Year + Mo n t h + D a y + H o u r + M i n u te + Second .$$

Compared with the prior art, the present disclosure has following beneficial effects.

First, the system integrates the sensor module, the processor module, the mechanical arm module, the defibrillator module, and the communication module into a whole, thereby improving the portability of the intelligent CPR defibrillator and simplicity of operation.

Second, the processor module uses an advanced algorithm to analyze the electrocardiogram signals, which may accurately judge conditions of the heart rate and the heart rhythm of the patients and improve accuracy of CPR demand judgment. The defibrillator module performs the automatic defibrillation when necessary, which is seamlessly connected with CPR operation, thereby improving timeliness and effectiveness of treatment.

Third, through the sensor module with high precision, the system may monitor the electrocardiogram signals of the patients in real time, and timely find emergency situations, such as sudden cardiac arrest. The mechanical arm module automatically performs the CPR according to the instruction from the processor module, which ensures stability and continuity of the CPR operation and is not affected by physical states of operators and different skill levels.

In view of problems of difficulty in quickly responding and poor process standardization in the actual use process. The present disclosure further provides a method suitable for automatically sending the operation time nodes by the intelligent CPR defibrillator as foregoing and applied to the system as foregoing. The method includes following steps.

S1: monitoring the electrocardiogram signals in real time by the sensor module.

S2: analyzing the electrocardiogram signals by the processor module and determining whether the CPR is needed.

S3: performing the CPR by the mechanical arm module if the CPR is needed.

S4: performing the automatic defibrillation by the defibrillator module if defibrillation is needed.

S5: recording the operation time nodes by the communication module and sending the operation time nodes to the remote monitoring system.

Furthermore, the S2 includes following steps.

S21: collecting consecutive electrocardiogram signal data ECG_Data.

S22: applying a filtering algorithm to remove noise to obtain a filtered electrocardiogram signal Filtered_ECG.

S23: identifying an R wave in the filtered electrocardiogram signal Filtered_ECG by a peak detection algorithm, and determining a heart rate Heart_Rate.

S24: analyzing a relationship between the Heart Rate and a preset normal heart rate range Normal_Heart_Rate_Range.

S25: triggering CPR demand assessment if the Heart Rate exceeds the Normal_Heart_Rate_Range.

Specifically, a calculation formula of the heart rate

$$Heart\_Rate = \frac{60}{Average\_RR \_Interval ( R\_Peaks )} .$$

The Average_RR_Interval (R_Peaks) is a function receiving a group of R wave peaks as input and calculating an average RR interval between the R wave peaks. The Average_RR_Interval (R_Peaks) first identifies all R wave peaks (R_Peaks) in the electrocardiogram signals, then measures a time interval between each pair of consecutive R waves, and finally calculates an average time interval, an RR interval refers to a time interval between two consecutive R wave peaks in the electrocardiogram signals, and a normal RR interval is regular and corresponds to a heartbeat cycle.

Furthermore, an automatic defibrillation algorithm applied to the S4 automatically adjusts electric shock energy according to the electrocardiogram signals, and specific steps are as follows.

S41: proceeding to a defibrillation preparation stage if a result of CPR_Necessity is true.

S42: measuring an amplitude ECG_Amplitude of the electrocardiogram signals.

S43: calculating required electric shock energy Shock_Energy according to the amplitude ECG_Amplitude with a formula of Shoe_Energy=k×ECG_Amplitude+b, where the k and the b are coefficients determined according to clinical data.

S44: adjusting the defibrillator module to an energy level required by the required electric shock energy Shock_Energy.

S45: performing electric shock, and recording electric shock time and energy at the same time for subsequent analysis.

Through the technical solution, compared with the prior art, the present disclosure has the following beneficial effects.

First, the method provides a set of standardized operation processes, including collecting and analyzing the electrocardiogram signals, and performing the CPR and the defibrillation, which ensures standardization and consistency of a treatment process.

Second, through real-time monitoring and rapid analysis, the method may quickly judge demand of the CPR, and greatly shorten a time from occurrence of the sudden cardiac arrest to a beginning of the treatment.

Third, automatic and intelligent CPR operation according to the method reduces human errors and improves a success rate of treatment on patients with the sudden cardiac arrest. At the same time, data recording and remote monitoring functions in the method provide important decision-making support information for medical staffs, which help optimize rescue strategies and improve overall medical service quality.

### BRIEF DESCRIPTION OF THE DRAWINGS

FIG. 1 is a schematic diagram of an overall structure according to the present disclosure.

FIG. 2 is a structural schematic diagram of a clamping plate according to the present disclosure.

FIG. 3 is a front schematic diagram of the present disclosure.

FIG. 4 is a block diagram of a system suitable for automatically sending operation time nodes by an intelligent cardiopulmonary resuscitation (CPR) defibrillator and applied to the intelligent CPR defibrillator of the present disclosure.

Reference numerals in the drawings: 100, constructing component; 101, constructing part; 101a, storage box; 101b, defibrillator body; 102, unfolding part; 102a, driving gear; 102b, driven gear; 102c, rotating rod; 102d, first connecting rod; 102e, at least one first placing box; 102f, second placing box; 102g, second connecting rod; 103, at least one clamping part; 103a, sliding rod; 103b, clamping plate; 103c, protective pad; 103d, fixing plate; 103e, return spring; 103f, toggle rod.

### DETAILED DESCRIPTION OF THE EMBODIMENTS

The embodiments of the present disclosure are described in further detail with reference to attached drawings and embodiments. The following embodiments are used to illustrate the present disclosure, rather than limit a scope of the present disclosure.

### Embodiment 1

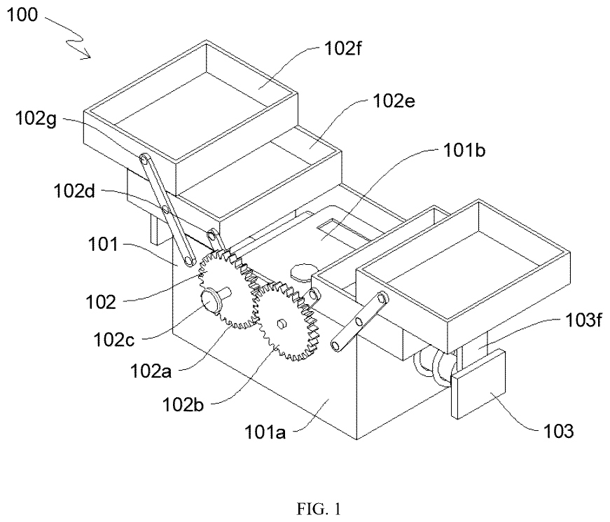

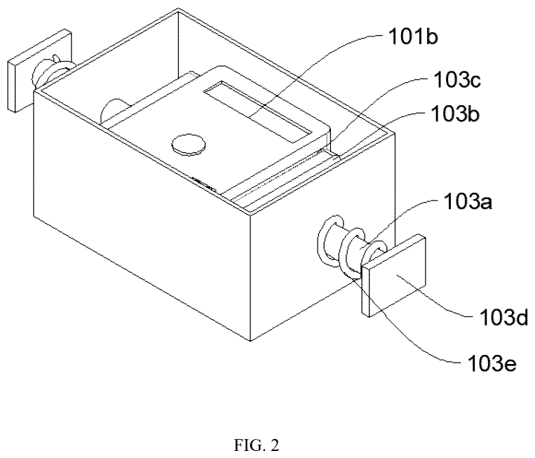

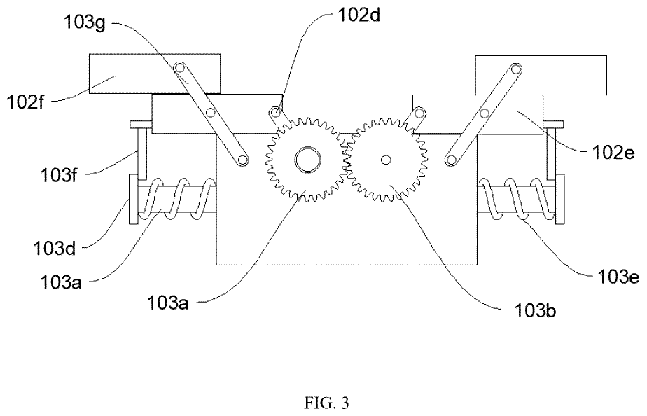

As shown in FIG. 1 to FIG. 3, the present disclosure provides an intelligent cardiopulmonary resuscitation (CPR) defibrillator, including a constructing component 100. The constructing component 100 includes a constructing part 101, an unfolding part 102 provided at a first side of the constructing part 101, and at least one clamping part 103 provided at a second side of the constructing part 101. The constructing part 101 includes a storage box 101a and a defibrillator body 101b provided in the storage box 101a. The at least one clamping part 103 includes a sliding rod 103a slidably provided at a first side of the storage box 101a, and a clamping plate 103b provided at one end of the sliding rod 103a.

Furthermore, the unfolding part 102 includes a driving gear 102a rotatably provided at a second side of the storage box 101a, a driven gear 102b rotatably provided at a first side of the driving gear 102a, and a rotating rod 102c provided at a second side of the driving gear 102a, the driving gear 102a is meshed with the driven gear 102b.

The unfolding part 102 further includes first connecting rods 102d respectively hinged at a third side of the driving gear 102a and a first side of the driven gear 102b, at least one first placing box 102e provided at a third side of the storage box 101a, a second placing box 102f provided at one side of the at least one first placing box 102e, and second connecting rods 102g provided at the second side of the storage box 101a, the second connecting rods 102a are respectively connected with a side wall of the storage box 101a, a side wall of the at least one first placing box 102e, and a side wall of the second placing box 102f. In one embodiment, two first placing boxes 102e and two second placing boxes 102 f are provided, when the driving gear 102a rotates, the driving gear drives the driven gear 102b to rotate and drives the first connecting rods 102d to swing, thereby driving the first placing boxes 102e at both sides to slide and unfold. When the two first placing boxes 102e slides and are unfolded, the two first placing boxes 102e respectively drive the second connecting rods 102g to swing to further drive the two second placing boxes 102f respectively disposed above the two first placing boxes 102e to unfold, thereby rapidly unfolding the two first placing boxes 102e and the two second placing boxes 102f when the defibrillator body 101b needs to be used.

Furthermore, the at least one clamping part 103 further includes a protective pad 103c provided at one end of the clamping plate 103b, a fixing plate 103d provided at one end of the sliding rod 103a, and a return spring 103e sleeved on an outer side of the sliding rod 103a. The at least one clamping part 103 further includes a toggle rod 103f provided at the first side of the storage box 101a. In one embodiment, the intelligent CPR defibrillator includes two clamping parts 103, two first placing boxes 102e, and two second placing boxes 102f, correspondingly, two sliding rods 103a, two clamping plates 103b, two protective pads 103c, two return springs 103e, and two toggle rods 103f are provided. In an initial state, the defibrillator body 101b is placed in the storage box 101a, the two clamping plates 103b together clamp, fix, and protect the defibrillator body 101b disposed therein through forces of the two return springs 103e. At the same time, the two protective pads 103c are provided to improve anti-skid effect. When the two first placing boxes 102e are unfolded to respectively drive the two second placing boxes 102f to unfold and slide outward, the two toggle rods 103f respectively at sides of the two first placing boxes 102e drive the two fixing plates 103d and the two sliding rods 103a to respectively slide to a direction of the storage box 101a, thereby driving the two clamping plates 103b to slide away from the defibrillator body 101b to release clamping and fixing on the defibrillator body 101b, in this way, the defibrillator body 101b is enabled to quickly remove for use.

Based on above, when the intelligent CPR defibrillator is in use, the rotating rod 102c rotates to drive the driving gear 102a to rotate, thereby driving the driven gear 102b to rotate and driving the first connecting rods 102d to swing, so as to drive the two first placing boxes 102e at both sides to slide and be unfolded. When the two first placing boxes 102e slide and unfold, the two first placing boxes respectively drive the second connecting rods 102g to swing and drive the two second placing boxes 102f disposed thereabove to fold, in this way, the two first placing boxes 102e and the two second placing boxes 102f are rapidly unfolded when the defibrillator body 101b needs to be used. When the two first placing boxes 102e and the two second placing boxes 102f are unfolded, the two toggle rods 103f respectively at the sides of the two first placing boxes 102e drive the two fixing plates 103d and the two sliding rods 103a to respectively slide to the direction of the storage box 101a, thereby driving the two clamping plates 103b to slide away from the defibrillator body 101b to release clamping and fixing on the defibrillator body 101b, in this way, the defibrillator body 101b is enabled to quickly remove for use.

### Embodiment 2

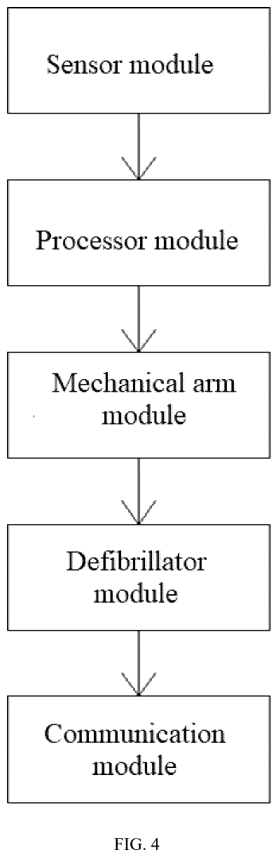

As shown in FIG. 1 to FIG. 4, differing from the embodiment 1, the embodiment 2 provides a system suitable for automatically sending operation time nodes by the intelligent CPR defibrillator and applied to the intelligent CPR defibrillator, including a sensor module, a processor module, a mechanical arm module, a defibrillator module, and a communication module.

The sensor module is configured to detect electrocardiogram signals of a human body and includes at least one bioelectrical signal sensor, where the at least one bioelectrical signal sensor is configured to monitor a heart rate and a heart rhythm of human body in real time.

The processor module is configured to analyze the electrocardiogram signals and determine whether CPR is needed.

The mechanical arm module is configured to perform the CPR.

The defibrillator module is configured to perform automatic defibrillation.

The communication module is configured to record the operation time nodes and automatically send the operation time nodes to a remote monitoring system.

Furthermore, the processor module employs a following algorithm formula to analyze the electrocardiogram signals and determine whether the CPR is needed,

$$CPR\_Necessity = \{ \begin{matrix}True if Heart\_Rate < Threshold\_Low or Heart\_Rate > \\ Threshold\_High or Heart\_Rhythm \_Irregularity \\ > Threshold\_Irregularity \\ False otherwise\end{matrix} .$$

Specifically, the CPR_Necessity is a Boolean value (True or False) determining whether the CPR is needed according to a comparison result of irregularity of the heart rate with a threshold of the heart rate and a comparison result of irregularity of the heart rhythm with a threshold of the heart rhythm.

The Heart_Rate is the heart rate, that is, the number of heart beats per minute, and is generally measured in beats per minute (bpm), which is determined by analyzing R wave peaks in the electrocardiogram signals. If any condition is met, the CPR_Necessity will be True, which indicates that the intelligent CPR defibrillator should start a CPR program.

The Threshold_LowThreshold_Low and the Threshold_HighThreshold_High are preset thresholds of the heart rate. If the heart rate is lower than Threshold Low or higher than Threshold_High, it may indicate that heart activity is abnormal and the CPR should be considered.

The Heart_Rhythm_IrregularityHeart_Rhythm_Irregularity is a measure of the irregularity of the heart rhythm, which is an indicator to measure changes of an RR interval, that is, a time interval between two consecutive R waves, in the electrocardiogram signals. The heart rhythm may indicate a heart disease or other medical emergencies. The Threshold_IrregularityThreshold_Irregularity is a threshold of the irregularity of the heart rhythm. If the Heart_Rhythm_Irregularity exceeds the threshold of the irregularity of the heart rhythm, it may indicate the heart rhythm is abnormal and the CPR should be considered.

Specifically, the mechanical arm module is configured to automatically adjust a position thereof and a force thereof according to an instruction from the processor module to implement effective CPR. The defibrillator module is configured to automatically adjust electric shock energy and perform electric shock when needing defibrillation.

The communication module is configured to send the operation time nodes to the remote monitoring system in real time in a format of

$$Time\_Stamp = Year + Mo n t h + D a y + H o u r + M i n u te + Second .$$

### Embodiment 3

As shown in FIG. 1 to FIG. 4, differing from the embodiments 1-2, the embodiment 3 provides a method suitable for automatically sending the operation time nodes by the intelligent CPR defibrillator and applied to the system, including following steps.

S1: monitoring the electrocardiogram signals in real time by the sensor module.

S2: analyzing the electrocardiogram signals by the processor module and determining whether the CPR is needed.

S3: performing the CPR by the mechanical arm module if the CPR is needed.

S4: performing the automatic defibrillation by the defibrillator module if defibrillation is needed.

S5: recording the operation time nodes by the communication module and sending the operation time nodes to the remote monitoring system.

Specifically, the communication module records and sends the operation time nodes to the remote monitoring system, and a format and a content of the operation time nodes are further defined as follows:

$$Time\_Stamp \_Message = \{ Time\_Stamp , Operation\_Type , Operation\_Result \} Time\_Stamp \_Message = \{ Time\_Stamp , Operation\_Type , Operation\_Result \} .$$

The Operation_Type indicates an operation type, such as the CPR or the defibrillation, and the Operation_Result indicates an operation result, such as success or failure.

Further, the S2 includes following steps.

S21: collecting consecutive electrocardiogram signal data ECG_Data.

S22: applying a filtering algorithm to remove noise to obtain a filtered electrocardiogram signal Filtered_ECG.

S23: identifying an R wave in the filtered electrocardiogram signal Filtered_ECG by a peak detection algorithm, and determining a heart rate Heart Rate.

S24: analyzing a relationship between the Heart_Rate and a preset normal heart rate range Normal_Heart_Rate_Range.

S25: triggering CPR demand assessment if the Heart_Rate exceeds the Normal_Heart_Rate_Range.

Specifically, a calculation formula of the heart rate is

$$Heart\_Rate = \frac{60}{Average\_RR \_Interval ( R\_Peaks )} .$$

An RR interval refers to a time interval between two consecutive R wave peaks in the electrocardiogram signals. R waves are one of the most prominent features in the electrocardiogram signals, which usually represents ventricular contraction of hearts. A normal RR interval is regular and corresponds to a heartbeat cycle.

The Average_RR_Interval (R_Peaks) is a function receiving a group of R wave peaks as input and calculating an average RR interval between the R wave peaks. The Average_RR_Interval (R_Peaks) first identifies all R wave peaks (R_Peaks) in the electrocardiogram signals, then measures a time interval between each pair of consecutive R waves, and finally calculates an average time interval.

The Heart_Rate refers to a heart rate generally expressing in beats per minute (bpm). The heart rate is calculated based on the RR interval, because one RR interval corresponds to a corresponding heartbeat cycle. Therefore, the heart rate is calculated by dividing 60 seconds by the average RR interval. Specific steps are as follows: detecting and identifying all R wave peaks from the electrocardiogram signals, measuring the time interval between each R wave peak and the next R wave peak to obtain a series of RR intervals, calculating the average of the series of the RR intervals, that is, the average RR interval, calculating the heart rate by the above formula; and dividing 60 by the average RR interval to obtain the number of beats per minute.

Furthermore, an automatic defibrillation algorithm applied to the S4 automatically adjusts electric shock energy according to the electrocardiogram signals, and specific steps are as follows.

S41: proceeding to a defibrillation preparation stage if a result of CPR_Necessity is true.

S42: measuring an amplitude ECG_Amplitude of the electrocardiogram signals.

S43: calculating required electric shock energy Shock_Energy according to the amplitude ECG_Amplitude with a formula of Shoe_Energy=k×ECG_Amplitude+b, where the k and the b are coefficients determined according to clinical data.

S44: adjusting the defibrillator module to an energy level required by the required electric shock energy Shock_Energy.

S45: performing electric shock, and recording electric shock time and energy at the same time for subsequent analysis.

The system further includes a data storage module storing data including device operation parameter data, device fault data, fault maintenance records, and maintenance solutions. The device fault data and the maintenance solutions are configured in a form of inspection diagrams. Once determining occurrence of a fault and identifying a type of the faucet, a fault processing module calls upon the data stored in the data storage module, finds out the corresponding device fault reasons, gives corresponding solutions, and initiating either manual or automatic maintenance. If no related cause or solution is found, only an alarm is given, the manual maintenance is required, and the fault and corresponding solutions are then recorded to enrich database of the data storage module.

The embodiments of the present disclosure are given for a purpose of illustration and description. Although the embodiments of the present disclosure have been shown and described above, it can be understood that the above embodiments are illustrative and cannot be understood as limitations of the present disclosure. Those who skilled in the art may make changes, modifications, substitutions and variations to the above embodiments within the scope of the present disclosure.

What is claimed is:

1. An intelligent cardiopulmonary resuscitation (CPR) defibrillator, comprising:
a constructing component;
wherein the constructing component comprises a constructing part, an unfolding part provided at a first side of the constructing part, and at least one clamping part provided at a second side of the constructing part;
the constructing part comprises a storage box and a defibrillator body provided in the storage box;
the at least one clamping part comprises a sliding rod slidably provided at a first side of the storage box, and a clamping plate provided at one end of the sliding rod.

2. The intelligent CPR defibrillator according to claim 1, wherein the unfolding part comprises a driving gear rotatably provided at a second side of the storage box, a driven gear rotatably provided at a first side of the driving gear, and a rotating rod provided at a second of the driving gear, and the driving gear is meshed with the driven gear; and
the unfolding part further comprises first connecting rods respectively hinged at a third side of the driving gear and a first side of the driven gear, at least one first placing box provided at a third side of the storage box, a second placing box provided at one side of the at least one first placing box, and second connecting rods provided at the second side of the storage box, the second connecting rods are respectively connected with a side wall of the storage box, a side wall of the at least one first placing box, and a side wall of the second placing box.

3. The intelligent CPR defibrillator according to claim 2, wherein the at least one clamping part further comprises a protective pad provided at one end of the clamping plate, a fixing plate provided at one end of the sliding rod, and a return spring sleeved on an outer side of the sliding rod; and
the at least one clamping part further comprises a toggle rod provided at the first side of the storage box.

4. A system, suitable for automatically sending operation time nodes by the intelligent CPR defibrillator and applied to the intelligent CPR defibrillator according to claim 1, comprising:
a sensor module, configured to detect electrocardiogram signals of a human body and including at least one bioelectrical signal sensor, wherein the at least one bioelectrical signal sensor is configured to monitor a heart rate and a heart rhythm of human body in real time;
a processor module, configured to analyze the electrocardiogram signals and determine whether CPR is needed;
a mechanical arm module, configured to perform the CPR;
a defibrillator module, configured to perform automatic defibrillation; and
a communication module, configured to record the operation time nodes and automatically send the operation time nodes to a remote monitoring system.

5. The system according to claim 4, wherein the processor module employs a following algorithm formula to analyze the electrocardiogram signals and determine whether the CPR is needed,
$$CPR\_Necessity = \{ \begin{matrix}True if Heart\_Rate < Threshold\_Low or Heart\_Rate > \\ Threshold\_High or Heart\_Rhythm \_Irregularity \\ > Threshold\_Irregularity \\ False otherwise\end{matrix}$$
the CPR_Necessity is a Boolean value (True or False) determining whether the CPR is needed according to a comparison result of irregularity of the heart rate with a threshold of the heart rate and a comparison result of irregularity of the heart rhythm with a threshold of the heart rhythm;
the Threshold_LowThreshold_Low and the Threshold_HighThreshold_High are preset thresholds of the heart rate, the Heart_Rhythm_IrregularityHeart_Rhythm_Irregularity is a measure of the irregularity of the heart rhythm, and the Threshold_IrregularityThreshold_Irregularity is a threshold of the irregularity of the heart rhythm.

6. The system according to claim 4, wherein the mechanical arm module is configured to automatically adjust a position thereof and a force thereof according to an instruction from the processor module to implement effective CPR;
the defibrillator module is configured to automatically adjust electric shock energy and perform electric shock when needing defibrillation.

7. The system according to claim 4, wherein the communication module is configured to send the operation time nodes to the remote monitoring system in real time in a format of
$$Time\_Stamp = Year + Mo n t h + D a y + H o u r + M i n u te + Second .$$

8. A method, suitable for automatically sending the operation time nodes by the intelligent CPR defibrillator and applied to the system according to claim 4, comprising:
S1: monitoring the electrocardiogram signals in real time by the sensor module;
S2: analyzing the electrocardiogram signals by the processor module and determining whether the CPR is needed;
S3: performing the CPR by the mechanical arm module if the CPR is needed;
S4: performing the automatic defibrillation by the defibrillator module if defibrillation is needed; and
S5: recording the operation time nodes by the communication module and sending the operation time nodes to the remote monitoring system.

9. The method according to claim 8, wherein the S2 comprises following steps:
S21: collecting consecutive electrocardiogram signal data ECG_Data;
S22: applying a filtering algorithm to remove noise to obtain a filtered electrocardiogram signal Filtered_ECG;
S23: identifying an R wave in the filtered electrocardiogram signal Filtered_ECG by a peak detection algorithm, and determining a heart rate Heart_Rate;
S24: analyzing a relationship between the Heart_Rate and a preset normal heart rate range Normal_Heart_Rate_Range; and
S25: triggering CPR demand assessment if the Heart_Rate exceeds the Normal_Heart_Rate_Range;
wherein a calculation formula of the heart rate is
$$Heart\_Rate = \frac{60}{Average\_RR \_Interval ( R\_Peaks )} ;$$
the Average_RR_Interval (R_Peaks) is a function receiving a group of R wave peaks as input and calculating an average RR interval between the R wave peaks; the Average_RR_Interval (R_Peaks) first identifies all R wave peaks (R_Peaks) in the electrocardiogram signals, then measures an time interval between each pair of consecutive R waves, and finally calculates an average time interval, an RR interval refers to an time interval between two consecutive R wave peaks in the electrocardiogram signals, and a normal RR interval is regular and corresponds to a heartbeat cycle.

10. The method according to claim 8, wherein an automatic defibrillation algorithm applied to the S4 automatically adjusts electric shock energy according to the electrocardiogram signals, and specific steps are as follows:
S41: proceeding to a defibrillation preparation stage if a result of CPR_Necessity is true;
S42: measuring an amplitude ECG_Amplitude of the electrocardiogram signals;
S43: calculating required electric shock energy Shock_Energy according to the amplitude ECG_Amplitude with a formula of Shoe_Energy=k×ECG_Amplitude+b, wherein the k and the b are coefficients determined according to clinical data;
S44: adjusting the defibrillator module to an energy level required by the required electric shock energy Shock_Energy; and
S45: performing electric shock, and recording electric shock time and energy at the same time for subsequent analysis.
